(19) United States
(12) Patent Application Publication
(10) Pub. No.: US 2018/0117141 A1
(43) Pub. Date: May 3, 2018

(54) DNA VACCINE COMPOSITION FOR PREVENTING AND TREATING HERPES ZOSTER, AND METHOD FOR ACTIVATING T CELLS FOR VZV ANTIGEN BY USING SAME

(71) Applicant: GENEONE LIFE SCIENCE CO., LTD., Seoul (KR)

(72) Inventors: Young Kun PARK, Blue Bell, PA (US); Byung Mun CHO, Incheon (KR); Moon Sup JEONG, Incheon (KR); Hyo Jin LEE, Seongnam-si, Gyeonggi-do (KR); Young Ran CHO, Seoul (KR); Joo Yeon OH, Seoul (KR)

(21) Appl. No.: 15/520,282

(22) PCT Filed: Jun. 16, 2015
(86) PCT No.: PCT/KR2015/006043

§ 371 Date: Apr. 19, 2017

(30) Foreign Application Priority Data
Oct. 21, 2014 (KR) 10-2014-0142531

(51) Int. Cl. A61K 39/25

(52) U.S. Cl. CPC A61K 39/25; A61K 2039/53

(57) ABSTRACT

The present invention provides a DNA vaccine composition for preventing and treating herpes zoster, containing: at least one type of plasmid containing the insertion site of a varicella-zoster virus (VZV)-derived gene encoding a VZV protein; and other pharmaceutically acceptable ingredients. The plasmid contains a plurality of plasmids containing the insertion site of heterologous genes which are different from each other. The plasmid contains: a first plasmid containing the insertion site of a first gene encoding IE-62 protein (SEQ ID NO: 1); a second plasmid containing the insertion site of a second gene encoding IE-63 protein (SEQ ID NO: 2); and a third plasmid containing the insertion site of a third gene encoding gE protein (SEQ ID NO: 3).

### TECHNICAL FIELD

The present invention relates to technology in the vaccination field, and more particularly, to a vaccine for preventing and treating herpes zoster which has excellent productivity, stability and immune reactivity against T cells and is applicable to various conditions of diseases.

### BACKGROUND ART

In general, herpes zoster is a skin rash disease that develops as a varicella-zoster virus (VZV) lies dormant in the neuronal ganglion until the immune system becomes weak, and then the reactivation of VZV is induced. When the herpes zoster develops, bullous lesions are formed, and patients have the aftereffects of postherpetic neuralgia even when the herpes zoster is restored. In this case, it is difficult to completely cure the postherpetic neuralgia once the postherpetic neuralgia develops, and thus the patients experience extreme pain. The reactivation of VZV and the onset of herpes zoster are associated with the weakening of cellular immune response in T cells. In particular, the herpes zoster is a disease that frequently develops in the aged or persons whose receive an immunosuppressive therapy.

When a patient develops herpes zoster, the patient will receive a treatment with an antiviral drug. However, this purpose of treatment is basically to prevent a rash from spreading to other sites by administering the antiviral drug within 72 hours after the patient begins to get a rash, and to reduce or alleviate the complications after the onset of herpes zoster. As the aged and immunosuppressed patients increase, the incidence rate of herpes zoster in Korea has increased rapidly, but there are no fundamental medications to treat the herpes zoster. Therefore, the herpes zoster is considered to be a disease that emphasizes the great importance of prophylactic vaccines.

Prophylactic vaccines against herpes zoster currently on the market have a medicinal effect approved in clinical trials, but the rate of decline in the onset of herpes zoster in response to the administration of the prophylactic vaccines reaches up to 50%. Therefore, there is a need for development of a novel vaccine against herpes zoster which has an excellent medicinal effect. Also, since the prophylactic vaccines against herpes zoster currently on the market are live attenuated vaccines, the prophylactic vaccines are in a paradoxical situation in which they cannot be rather used for immunosuppressed patients having a high incidence rate of herpes zoster. Accordingly, there is an urgent demand for development of a novel vaccine against herpes zoster which can be safely administered to the immunosuppressed patients.

### DISCLOSURE

### Technical Problem

Therefore, the present invention is designed to solve the problems of the prior art, and it is an object of the present invention to provide a DNA vaccine composition for preventing and treating herpes zoster, which is capable of being applied to patients having various types of diseases and has remarkably improved stability.

It is another object of the present invention to provide a method for activating T cells against a VZV antigen by effectively administering the DNA vaccine composition for preventing and treating herpes zoster into the body.

### Technical Solution

According to an aspect of the present invention, there is provided a plasmid DNA for preventing and treating herpes zoster, which contains an insertion site of a varicella-zoster virus (VZV)-derived gene encoding a VZV protein, wherein the plasmid DNA is directly administered into the body by an electroporation method to induce immune activity of T cells against a VZV antigen.

An electroporation system (CELLECTRA commercially available from Inovio Pharmaceuticals Inc.) was used as an electroporation system for the electroporation.

The gene may include one selected from the group consisting a first gene encoding an IE-62 protein (SEQ ID NO: 1), a second gene encoding an IE-63 protein (SEQ ID NO: 2), and a third gene encoding a gE protein (SEQ ID NO: 3).

Specifically, the plasmid may include one selected from the group consisting a plasmid DNA set forth in SEQ ID NO: 4, a plasmid DNA set forth in SEQ ID NO: 5, and 1 plasmid DNA set forth in SEQ ID NO: 6.

According to another aspect of the present invention, there is provided a DNA vaccine composition for preventing and treating herpes zoster, which includes at least one plasmid containing an insertion site of a varicella-zoster virus (VZV)-derived gene encoding a VZV protein, and other pharmaceutically acceptable ingredients. The other ingredients may, for example, include water such as saline, etc.

The DNA vaccine composition may include a plurality of plasmids, which contain different insertion sites of heterologous genes, as the plasmid constituting the DNA vaccine. For example, a plurality of plasmids containing different genetic loci encoding the VZV protein may be used as the plasmid.

Three plasmids such as a first plasmid containing an insertion site of a first gene encoding an IE-62 protein (SEQ ID NO: 1), a second plasmid containing an insertion site of a second gene encoding an IE-63 protein (SEQ ID NO: 2), and a third plasmid containing an insertion site of a third gene encoding a gE protein (SEQ ID NO: 3) may be used as the plasmid included in the DNA vaccine composition. A content of each of the plasmids in the composition may be variously adjusted in consideration of the type of a disease, the age of a subject to which the plasmid is administered, etc.

According to still another aspect of the present invention, there is provided a method for activating T cells against a varicella-zoster virus (VZV) antigen, which includes preparing a plasmid containing a VZV-derived gene encoding a VZV protein, and administering the plasmid into the body using an electroporation method. In this case, the plasmid may be mass-produced using a mass production system.

### Advantageous Effects

The DNA vaccine for preventing and treating herpes zoster according to the present invention does not cause safety issues caused by live attenuated vaccines since the DNA vaccine is a vaccine delivered through direct administration of a plasmid. Also, the plasmid can be easily mass-produced, and stability of the vaccine during a distribution process can be remarkably improved. Since the DNA vaccine is administered into the body using an electroporation method, the DNA vaccine can exhibit remarkable in vivo delivery efficiency, compared to conventional injectable drugs. Meanwhile, the DNA vaccine for preventing and treating herpes zoster can be used for a therapeutic purpose as well as a prophylactic purpose, and may be properly used for patients having various diseases, and patients of different ages. The DNA vaccine for preventing and treating herpes zoster has an advantage in that personalized vaccines can be designed through various combinations of a plurality of plasmids containing different insertion sites of plasmid genes.

### DESCRIPTION OF DRAWINGS

FIG. 1 is a diagram showing a vaccination schedule according to one exemplary embodiment of the present invention.

FIG. 2 shows graphs illustrating results of immunogenicity for experimental groups according to one exemplary embodiment of the present invention.

FIGS. 3 and 4 are graphs illustrating results of comparing immune responses of overlapping peptides of proteins in respective experimental groups.

FIGS. 5 to 7 are electrophoretic image of products expressed by respective plasmid genes.

### BEST MODE

Hereinafter, a DNA vaccine for preventing and treating herpes zoster will be described in detail.

The DNA vaccine for preventing and treating herpes zoster according to the present invention is not a virus-based live vaccine but a DNA-based vaccine in which DNA itself is administered into the body. As a result, the DNA itself in the form of a plasmid, which is administered into the body to induce immune activity of T cells against a VZV antigen, serves as the vaccine.

The DNA vaccine encodes a VZV-derived antigen protein, and ultimately plays a role in forming the antigen protein in the body. The VZV-derived protein is preferably chosen in consideration of desired characteristics of the DNA vaccine. In this case, the protein may be chosen alone or in combination with plural types thereof.

According to one exemplary embodiment of the present invention, the protein may include proteins IE-62 (SEQ ID NO: 1) and IE-63 (SEQ ID NO: 2) and a glucoprotein gE (SEQ ID NO: 3), all of which may induce a cellular immune response, and the plasmid is designed so that a gene encoding such a protein can be inserted into the plasmid.

The plasmid DNA is administered so that the plasmid DNA is mixed with a liquid in the body. For this purpose, the plasmid DNA of the present invention may be designed to be introduced into the body with high efficiency using an electroporation method. Only when such an electroporation method is used, in vivo delivery efficiency may be maximized, thereby maximizing therapeutic efficacy. A site of the body to which the plasmid DNA is administered using the electroporation method includes a region of muscle, etc., but the present invention is not particularly limited thereto. For example, the site of the body may be determined in consideration of various factors such as the age of a subject to which the plasmid DNA is administered, the type of a disease, concurrent diseases, etc. An electroporation system (CELLECTRA commercially available from Inovio Pharmaceuticals Inc.) may be used as electroporation equipment used to realize the electroporation method.

The genes, for example, a first gene, a second gene and a third gene which may encode and express the IE-62, IE-63 and gE proteins, respectively, are incorporated into the plasmid using a conventional method. However, to maximize the medicinal effect and administration efficiency, plasmids set forth, respectively, in SEQ ID NO: 4, SEQ ID NO: 5 and SEQ ID NO: 6 may be designed in this example to maximize the efficiency of the DNA vaccine. The respective plasmids may be used alone, but are preferably included in the DNA vaccine in a combination of two or more different plasmids. For example, the DNA vaccine containing all the three plasmids may be contemplated in one exemplary embodiment of the present invention. Meanwhile, different types of plasmids which are not specified in this example may also be newly introduced.

The plasmid may be administered into the body by means of an injectable drug, etc. In this case, the plasmid should be prepared with a high concentration and high purity to improve delivery efficiency and immune response efficiency. Also, the ease in mass production should be ensured in a commercial aspect. The plasmid according to one exemplary embodiment of the present invention has a foundation to be mass-produced with such a high concentration and high purity.

The DNA vaccine for preventing and treating herpes zoster according to one exemplary embodiment of the present invention is a vaccine for liquids, that is, injectable drugs, which includes at least one plasmid DNA as described above. Thus, the DNA vaccine includes the plasmid DNA and a liquid ingredient. Here, the liquid ingredient may be pure water. In addition to the water, the DNA vaccine composition according to one exemplary embodiment of the present invention may further include pharmaceutically acceptable functional additives. In this case, such additives may be introduced at a level known in the related art.

The DNA vaccine composition may, for example, include all the three plasmids as described above.

Hereinafter, the efficiency of the plasmid DNA according to one exemplary embodiment of the present invention will be described based on the specific contents of experiments.

### [Experiment 1] Evaluation of Immunogenicity

Screening of VZV Gene Candidates Usable in DNA Vaccine

Genes of proteins IE-62 and IE-63 and a glucoprotein gE, all of which may induce a cellular immune response, were screened from 67 genes encoding proteins in the VZV genome. The type of the VZV genome was divided into a total of 5 clades, and there was no significant difference in amino acid sequences of the IE-62, IE-63 and gE between the clades. Plasmids were constructed using sequences of the aforementioned IE-62, IE-63 and gE genes. The amino acid sequences of the proteins are as set forth in SEQ ID NOs: 1 to 3, and the DNA sequences of the constructed plasmids are as set forth in SEQ ID NOs: 4 to 6. In this example, the DNA vaccine included the plasmid and a liquid ingredient (i.e., water), but did not include other ingredients.

Experiment on Mice

The IE-62, IE-63 and gE genes were prepared as candidate materials for the DNA vaccine, and administered into C57BL/6 mice. Thereafter, splenocytes were removed from the mice to evaluate immunogenicity of T cells against a VZV antigen. A total of 5 groups were used for this experiment, and each group consisted of five mice, as follows.

1) Neg: A group in which mice are vaccinated with a vector (n=5)

2) IE62: A group in which mice are vaccinated with an IE-62 DNA vaccine (n=5)

3) IE63: A group in which mice are vaccinated with an IE-63 DNA vaccine (n=5)

4) gE: A group in which mice are vaccinated with a gE DNA vaccine (n=5)

5) IE62+IE63+gE: A group in which mice are vaccinated with a mixture of IE-62, IE-63 and gE DNA vaccines

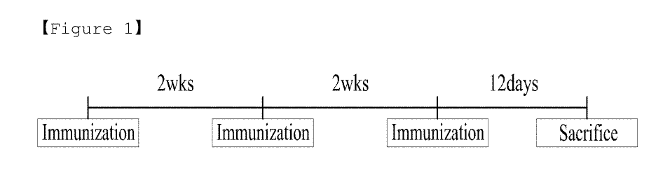

The vaccination was performed three times at an interval of 2 weeks using an electroporation system (CELLECTRA commercially available from Inovio Pharmaceuticals Inc.). In this case, an amount of the DNA was 30 μg/mice upon every vaccination, and the mixed group was vaccinated at 30 μg for each DNA (a total amount of 90 μg). After 12 days of the vaccination, splenocytes were removed to perform an immuoassay. A vaccination schedule is shown in FIG. 1. FIG. 1 is a diagram showing a vaccination schedule according to one exemplary embodiment of the present invention.

Preparation of Antigen for Experiments

To systematically measure the entire immune response to proteins encoded by candidate genes, a series of overlapping peptides (OLPs) were designed and prepared. Each of the overlapping peptides was designed to consist of 15 amino acids with 10 amino acids overlapping with its adjacent peptides. Also, the overlapping peptides were designed to contain full-length amino acid sequences of the IE-62, IE-63 and gE proteins. As a result, 261 IE-62 overlapping peptides, 54 IE-63 overlapping peptides and 124 gE overlapping peptides were prepared. The 37 or 38 consecutive IE-62 overlapping peptides were mixed to prepare a total of 7 peptide mixtures, which were then used. The 27 consecutive IE-63 overlapping peptides were mixed to prepare a total of 2 peptide mixtures, which were then used. The 40 or 41 consecutive IE-63 overlapping peptides were mixed to prepare a total of 3 peptide mixtures, which were then used. 5% DMSO was used as the negative control, and a mixture of phorbol myristate acetate (PMA) and ionomycin was used as the positive control. Peptide mixtures prepared by mixing the overlapping peptides are listed in the following Table 1.

TABLE 1

| | | | Number of mixed |
|---|---|---|---|
| | Antigen | SEQ ID NOs of | overlapping |
| Proteins | names | amino acids | peptides |
| IE-62 | IE62-1 | 1-37 | 37 |
| | IE62-2 | 28-74 | 37 |
| | IE62-3 | 75-111 | 37 |
| | IE62-4 | 112-143 | 37 |
| | IE62-5 | 149-185 | 37 |
| | IE62-6 | 186-223 | 38 |
| | IE62-7 | 224-261 | 38 |
| IE-63 | IE63-1 | 1-27 | 27 |
| | IE63-2 | 28-54 | 27 |
| gE | gE -1 | 1-27 | 41 |
| | gE -2 | 28-54 | 40 |
| | gE -3 | 1-41 | 41 |

A lysate prepared after MRC-5 cells were infected with VZV was used as the antigen, and an MRC-5 cell lysate was used as a negative antigen corresponding to the lysate.

IFN-γ ELISPOT Assay Protocol Splenocytes were seeded at 1×10⁶ cells/well to perform an ELISPOT assay. When the splenocytes were stimulated with a mixture of overlapping peptides, the overlapping peptides were administered so that a concentration of each of the overlapping peptides reached 1 μg/ml per well, and, when the splenocytes were stimulated with a lysate antigen, the lysate antigen was administered so that a concentration of the lysate antigen reached 50 μg/ml per well. In this case, this experiment was repeated in triplicate. The assay results obtained through the stimulation with the overlapping peptide antigen was calculated, as follows: Mean of OLP pools-specific spot forming cells (SFCs)-Mean of 5% DMSO-specific SFCs. The assay results obtained through the stimulation with the lysate antigen was calculated, as follows: Mean of VZV-specific SFCs-Mean of MRC-5-specific SFCs. The maximum value of SFCs per well was calculated to be 500.

Experimental Results of Immunogenicity

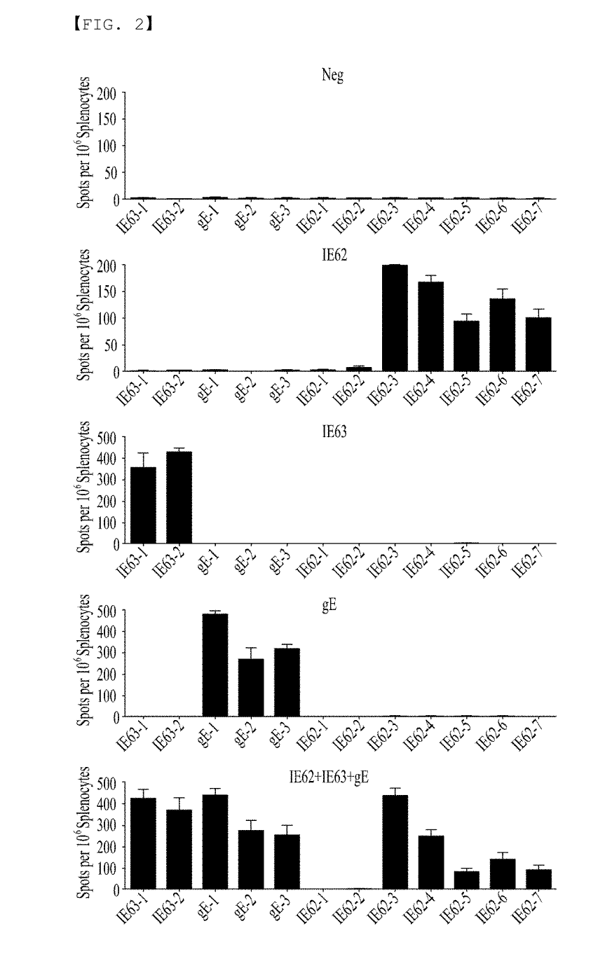

The results of antigen evaluation with respect to the respective vaccines showed that no immune response was induced with respect to the stimulus of overlapping peptides in the Neg-treated group used as the negative control. It was revealed that an immune response was induced with respect to the overlapping peptide of the corresponding protein in the IE-62-, IE-63- and gE-treated groups in which each of the IE-62, IE-63 and gE DNA vaccines were administered alone. It was revealed that an immune response was induced with respect to the overlapping peptides of the three proteins in the IE62+IE63+gE-treated group in which all the IE-62, IE-63 and gE DNA vaccines were administered. When a DNA vaccine of a candidate material was administered, an immune response was induced with respect to the overlapping peptides specific to the candidate material. FIG. 2 shows graphs illustrating results of immunogenicity for experimental groups according to one exemplary embodiment of the present invention.

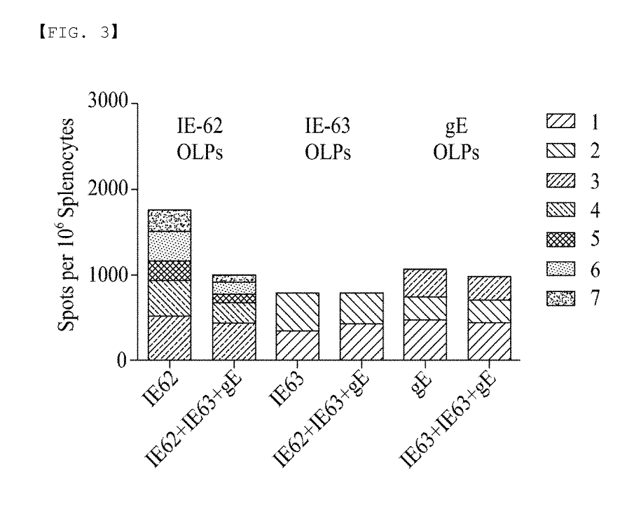

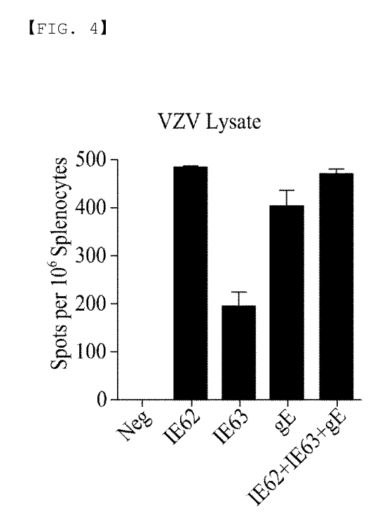

FIGS. 3 and 4 are graphs illustrating results of comparing immune responses of overlapping peptides of proteins in respective experimental groups. Referring to FIG. 3, the immune responses of the overlapping peptides of the corresponding proteins were compared in the groups in which a single DNA vaccine was administered and the group in which a mixture of three DNA vaccines was administered. As a result, it was revealed that, when the mixture of three DNA vaccines was administered, the immune response to IE-62 decreased by 43.3%, there was no difference in the immune response to IE-63, and the immune response to gE decreased by 9.6%, compared to when the single DNA vaccine was administered.

Validity of Selection of Antigen

Referring to FIG. 4, the splenocytes were stimulated with the lysate antigen in each of the experimental groups. As a result, it was confirmed that no immune response was induced in the Neg-treated group, but a strong immune response was induced in the group in which the DNA vaccine of the candidate material was administered. From these result, it can be seen that the proteins such as IE-62, IE-63, gE and the like were expressed in the cells infected with VZV, and an immune response occurred in the cells infected with VZV when the immune response to IE-62, IE-63 and gE selected as the candidate materials was induced in this experiment.

Experimental Evaluation

In a laboratory animal model, the DNA vaccines prepared using the genes encoding the IE-62, IE-63 and gE proteins were evaluated and analyzed. As a result, it was revealed that the T cells exhibited sufficient immunogenicity when the DNA vaccines administered alone or in combination thereof. The immunogenicity of the T cells was analyzed through the stimulation of the VZV lysate antigen. As a result, it was judged that it was reasonable to select the IE-62, IE-63 and gE as the candidate materials for the vaccines.

### [Experiment 2] Evaluation of In Vitro Protein Expression

Criteria for Expression Confirmation
Transfection: RD cells (1×10⁶ cells/T75 flask), 30 μg of pDNA, Lipofectamine 2000, cell harvesting after 24 hours of transfection
RD cells: human rhabdomyosarcoma
Cell lysis: 2×10⁶ cells/90 μl of RIPA buffer with Protease Inhibitor Cocktail
SDSPAGE: LDS sample buffer with Reducing agent (DTT), 30 μl loading (19.5 μl of lysate), 4 to 12% Bis-Tris gel, MES running buffer at 165 V for 35 minutes
Protein transfer: NC membrane, Dry blotting at 20 V for 1 minute→at 23 V for 4 minutes→at 25 V for 2 minutes
Protein detection: 1) Blocking for 1 hour, 2) Antibody reaction (1:100) for an hour and a half (HA-probe HRP (SantaCruz, Cat. No.: sc-805HRP), Goat anti-VZV IE62 (SantaCruz, Cat. No.: sc-17525), Donkeyanti-goat Ig GHRP (SantaCruz, Cat. No.: sc-2020), 3), and HRP chromogenic substrate (Invitrogen, Cat. No.: WP20004))

Evaluation of Expression Confirmation

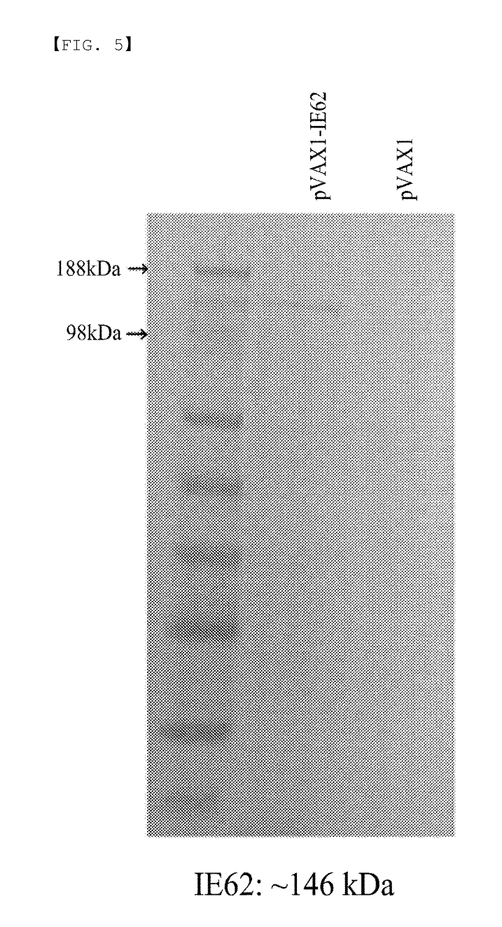

FIGS. 5 to 7 are electrophoretic image of products expressed by the respective plasmid genes.

Referring to FIG. 5, the plasmid encoding an IE-62 gene was introduced into RD cells derived from human rhabdomyosarcoma using Lipofectamine 2000. After 24 hours, the cells were lysed to extract proteins, and the proteins were then subjected to Western Blotting using an antibody for detecting an expressed antigen. An expressed protein of the plasmid encoding the IE-62 gene was detected using anti-VZV IE62 (SantaCruz, Cat. No.: SC-2020), and visualized using a HRP chromogenic substrate (Invitrogen, Cat. No.: WP20004). As a result, it was revealed that the product size of the insert gene expressed from the plasmid encoding the IE-62 gene was identical to the predicted molecular weight of the insert gene, indicating that the product of the gene introduced into the RD cells was the IE-62 antigen protein of VZV.

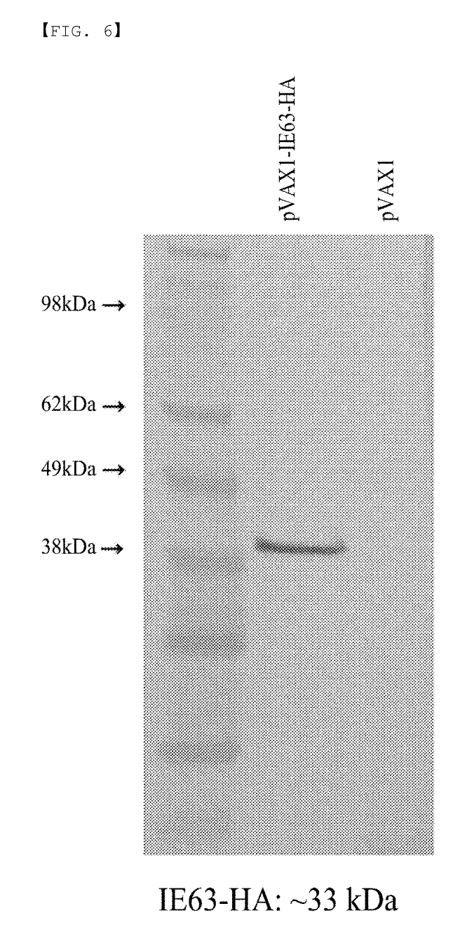

Referring to FIG. 6, the plasmid encoding an IE-63 gene was introduced into RD cells derived from human rhabdomyosarcoma using Lipofectamine 2000. After 24 hours, the cells were lysed to extract proteins, and the proteins were then subjected to Western Blotting using an antibody for detecting an expressed antigen. An expressed protein of the plasmid encoding the IE-63 gene was detected using HA-probe HRP (SantaCruz, Cat. No.: SC-805HRP), and visualized using a HRP chromogenic substrate (Invitrogen, Cat. No.: WP20004). As a result, it was revealed that the product size of the insert gene expressed from the plasmid encoding the IE-63 gene was identical to the predicted molecular weight of the insert gene, indicating that the product of the gene introduced into the RD cells was the IE-63 antigen protein of VZV.

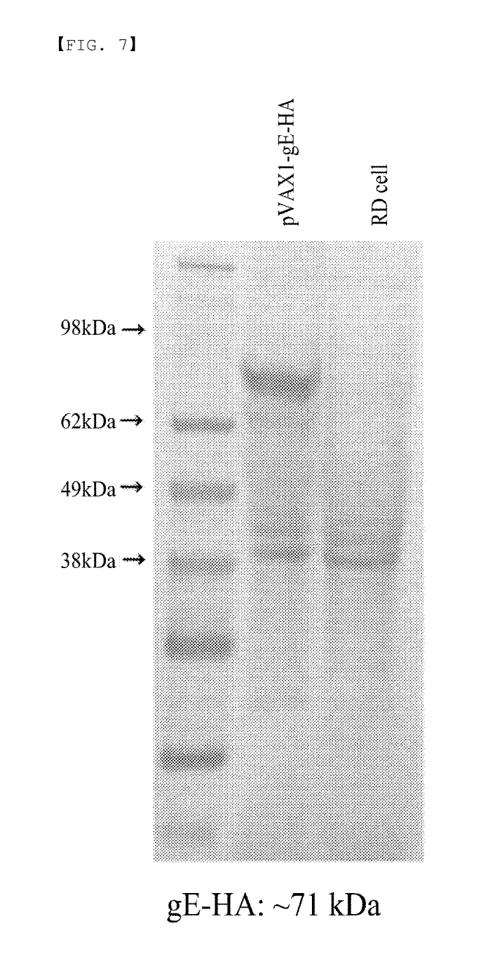

Referring to FIG. 7, the plasmid encoding a gE gene was introduced into RD cells derived from human rhabdomyosarcoma using Lipofectamine 2000. After 24 hours, the cells were lysed to extract proteins, and the proteins were then subjected to Western Blotting using an antibody for detecting an expressed antigen. An expressed protein of the plasmid encoding the gE gene was detected using HA-probe HRP (SantaCruz, Cat. No.: SC-805HRP), and visualized using a HRP chromogenic substrate (Invitrogen, Cat. No.: WP20004). As a result, it was revealed that the product size of the insert gene expressed from the plasmid encoding the gE gene was identical to the predicted molecular weight of the insert gene, indicating that the product of the gene introduced into the RD cells was the gE antigen protein of VZV.

1. A plasmid DNA for preventing and treating herpes zoster, comprising an insertion site of a varicella-zoster virus (VZV)-derived gene encoding a VZV protein,
wherein the plasmid DNA is directly administered into the body by an electroporation method to induce immune activity of T cells against a VZV antigen.

2. The plasmid DNA of claim 1, wherein the gene comprises one selected from the group consisting of a first gene encoding an IE-62 protein (SEQ ID NO: 1), a second gene encoding an IE-63 protein (SEQ ID NO: 2), and a third gene encoding a gE protein (SEQ ID NO: 3).

3. The plasmid DNA of claim 1, which comprises one selected from the group consisting of a plasmid DNA set forth in SEQ ID NO: 4, a plasmid DNA set forth in SEQ ID NO: 5, and a plasmid DNA set forth in SEQ ID NO: 6.

4. A DNA vaccine composition for preventing and treating herpes zoster, comprising:
at least one plasmid containing an insertion site of a varicella-zoster virus (VZV)-derived gene encoding a VZV protein; and
other pharmaceutically acceptable ingredients.

5. The DNA vaccine composition of claim 4, wherein the plasmid comprises a plurality of plasmids containing different insertion sites of heterologous genes.

6. The DNA vaccine composition of claim 4, wherein the plasmid comprises:
a first plasmid containing an insertion site of a first gene encoding an IE-62 protein (SEQ ID NO: 1);
a second plasmid containing an insertion site of a second gene encoding an IE-63 protein (SEQ ID NO: 2); and
a third plasmid containing an insertion site of a third gene encoding a gE protein (SEQ ID NO: 3).

7. A method for activating T cells against a varicella-zoster virus (VZV) antigen, comprising:
preparing a plasmid containing a VZV-derived gene encoding a VZV protein; and
administering the plasmid into the body using an electroporation method.


SEQUENCE LISTING (6 sequences)

SEQ ID NO: 1 - protein, 1328 aa, Varicella-Zoster Virus
MDWTWILFLVAAATRVHSMDTPPMQRSTPQRAGSPDTLELMDLLDAAAAAAEHRARVVTSSQPDDLLFGENGVMVGREHEIVSIPSVSGLQPEPRTEDVGEELTQDDYVCEDGQDLMGSPVIPLAEVFHTRFSEAGAREPTGADRSLETVSLGTKLARSPKPPMNDGETGRGTTPPFPQAFSPVSPASPVGDAAGNDQREDQRSIPRQTTRGNSPGLPSVVHRDRQTQSISGKKPGDEQAGHAHASGDGVVLQKTQRPAQGKSPKKKTLKVKVPLPARKPGGPVPGPVEQLYHVLSDSVPAKGAKADLPFETDDTRPRKHDARGITPRVPGRSSGGKPRAFLALPGRSHAPDPIEDDSPVEKKPKSREFVSSSSSSSSWGSSSEDEDDEPRRVSVGSETTGSRSGREHAPSPSNSDDSDSNDGGSTKQNIQPGYRSISGPDPRIRKTKRLAGEPGRQRQKSFSLPRSRTPIIPPVSGPLMMPDGSPWPGSAPLPSNRVRFGPSGETREGHWEDEAVRAARARYEASTEPVPLYVPELGDPARQYRALINLIYCPDRDPIAWLQNPKLTGVNSALNQFYQKLLPPGRAGTAVTGSVASPVPHVGEAMATGEALWALPHAAAAVAMSRRYDRAQKHFILQSLRRAFASMAYPEATGSSPAARISRGHPSPTTPATQTPDPQPSAAARSLSVCPPDDRLRTPRKRKSQPVESRSLLDKIRETPVADARVADDHVVSKAKRRVSEPVTITSGPVVDPPAVITMPLDGPAPNGGFRRIPRGALHTPVPSDQARKAYCTPETIARLVDDPLFPTAWRPALSFDPGALAEIAARRPGGGDRRFGPPSGVEALRRRCAWMRQIPDPEDVRLLIIYDPLPGEDINGPLESTLATDPGPSWSPSRGGLSVVLAALSNRLCLPSTHAWAGNWTGPPDVSALNARGVLLLSTRDLAFAGAVEYLGSRLASARRRLLVLDAVALERWPRDGPALSQYHVYVRAPARPDAQAVVRWPDSAVTEGLARAVFASSRTFGPASFARIETAFANLYPGEQPLCLCRGGNVAYTVCTRAGPKTRVPLSPREYRQYVLPGFDGCKDLARQSRGLGLGAADFVDEAAHSHRAANRWGLGAALRPVFLPEGRRPGAAGPEAGDVPTWARVFCRHALLEPDPAAEPLVLPPVAGRSVALYASADEARNALPPIPRVMWPPGFGAAETVLEGSDGTRFVFGHHGGSERPAETQAGRQRRTADDREHALEPDDWEVGCEDAWDSEEGGGDDGDAPGSSFGVSIVSVAPGVLRDRRVGLRPAVKVELLSSSSSSEDEDDVWGGRGGRSPPQSRG

SEQ ID NO: 2 - protein, 296 aa, Varicella-Zoster Virus
MDWTWILFLVAAATRVHSMFCTSPATRGDSSESKPGASVDVNGKMEYGSAPGPLNGRDTSRGPGAFCTPGWEIHPARLVEDINRVFLCIAQSSGRVTRDSRRLRRICLDFYLMGRTRQRPTLACWEELLQLQPTQTQCLRATLMEVSHRPPRGEDGFIEAPNVPLHRSALECDVSDDGGEDDSDDDGSTPSDVIEFRDSDAESSDGEDFIVEEESEESTDSCEPDGVPGDCYRDGDGCNTPSPKRPQRAIERYAGAETAEYTAAKALTALGEGGVDWKRRRHEAPRRHDIPPPHGV

SEQ ID NO: 3 - protein, 641 aa, Varicella-Zoster Virus
MDWTWILFLVAAATRVHSMGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 4 - DNA, 6951 bp, Artificial Sequence
  pGX-5101
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggattctgttcctggtggccgctgccacacgggtgcacagcatggataccccccccatgcagagaagcacccctcagagagccggcagccccgacaccctggaactgatggatctgctggatgccgccgctgctgccgccgaacatagagccagagtggtcaccagcagccagcccgacgatctgctgttcggcgagaacggcgtgatggtcggaagagaacacgagatcgtgtccatccccagcgtgtccggactgcagcctgagcccagaacagaggacgtgggcgaggaactgacccaggacgactacgtgtgcgaggacggccaggatctgatgggcagccctgtgattcccctggccgaggtgttccacaccagattcagcgaggcaggcgccagagagcccacaggcgccgatagaagcctggaaaccgtgtccctgggcaccaagctggccagatcccccaagccccccatgaacgatggcgagacaggcagaggcaccaccccaccattccctcaggccttctctcctgtgtcccctgccagcccagtgggcgacgctgccggaaacgatcagagagaggaccagcggagcatccccagacagaccaccagaggcaatagccctggcctgcctagcgtggtgcaccgggatagacagacccagagcatcagcggcaagaagccaggcgacgaacaggccggacatgcccatgctagcggagatggcgtggtgctgcagaaaacccagaggcctgcccagggcaagagccccaagaaaaagaccctgaaagtgaaggtgcccctgcccgccagaaagcctggtggacctgtgcctggacccgtggaacagctgtatcacgtgctgagcgacagcgtgccagccaagggcgccaaggccgatctgcccttcgagacagacgacacccggcccagaaagcacgacgccagaggcatcacacccagagtgccaggcagaagctccggcggcaagcctagagcctttctggccctgcccggaagaagccacgcccccgatcctatcgaggacgacagccccgtggaaaagaagcccaagagccgcgagttcgtgtcctccagcagcagctctagcagctggggcagctccagcgaggacgaggacgatgagcctagaagagtgtccgtgggcagcgagacaaccggcagcagatctggcagagagcacgcccccagccccagcaactccgacgacagcgatagcaatgacggcggctccaccaagcagaacatccagcctggctaccggtccatctccggccctgacccccggatcagaaagaccaagagactggccggcgagcccggcagacagagacagaagtccttcagcctgccccggtccagaacccccatcatccctccagtgtccggccctctgatgatgcccgatggctctccttggcctggcagcgcccctctgccctccaatagagtcagattcggccccagcggcgagacaagagagggacattgggaggacgaagccgtgcgggctgccagagccagatacgaggcctctacagagcccgtgcctctgtacgtgccagagctgggagatcccgccagacagtaccgggccctgatcaacctgatctactgccccgaccgggaccccattgcctggctgcagaatcctaagctgaccggcgtgaacagcgccctgaaccagttctaccagaagctgctgcctcctggcagagccggaacagccgtgacaggatctgtggcctctccagtgcctcacgtgggagaagccatggccacaggcgaagctctgtgggccctgcctcatgctgctgcagctgtggctatgagcagaagatacgaccgcgcccagaagcacttcatcctgcagagcctgcggagagccttcgccagcatggcctatcctgaggccaccggatctagcccagccgccagaatcagcagaggccaccctagccctaccacccctgccacacagacccctgatcctcagcctagcgctgccgccagatctctgagcgtgtgcccccctgacgacagactgcggacccctagaaagcggaagtcccagcccgtggaatcccggtccctgctggacaagatcagagaaacccccgtggccgacgccagggtggccgatgatcatgtggtgtccaaggccaagcggcgggtgtccgagcctgtgacaatcacaagcggccctgtggtggacccccctgccgtgatcacaatgccactggacggacctgcccccaacggcggcttcagaagaattcctagaggcgccctgcacacccccgtgcctagcgatcaggccagaaaagcctactgcacccccgagacaatcgccagactggtggacgaccctctgttcccaacagcctggcggcctgccctgtcttttgatcctggcgctctggccgaaatcgccgctagaaggccaggcggaggcgacagaagatttggccctcctagcggcgtggaagccctgaggcgaagatgcgcctggatgagacagatccccgaccccgaagatgtgcggctgctgatcatctacgaccccctgcctggcgaggacatcaacggcccactggaaagcaccctggccaccgaccctggaccatcctggtcaccttctagaggcggactgtctgtggtgctggccgccctgagcaacagactgtgtctgccttctacacacgcctgggccggcaattggacaggccctcctgatgtgtccgccctgaatgccagaggcgtcctgctgctgtctacccgggatctcgctttcgctggcgccgtggaatacctgggctctagactggctagcgctcggcggagactgctggtcctggatgctgtggccctggaacggtggcctagagatggcccagccctgagccagtaccatgtgtatgtgcgcgctcccgccaggcctgatgcacaggcagtcgtcagatggcccgattctgccgtgaccgaaggactggccagagccgtgtttgccagctccagaacattcggcccagccagcttcgcccggatcgagacagcctttgccaacctgtaccccggcgagcagcccctgtgtctgtgtagaggcggcaacgtggcctacaccgtgtgtaccagagccggccctaagaccagggtgccactgagccctcgggaataccggcagtacgtgctgcccggcttcgacggctgtaaagacctggccagacagagcaggggcctgggactgggagccgctgattttgtggatgaggccgcccacagccacagagccgccaatagatggggactgggcgctgctctgaggcccgtgtttctgccagagggtagacgaccaggcgccgctggacctgaagcaggggatgtgcctacctgggccagagtgttttgtcggcacgctctgctggaacccgatccagccgctgaacctctggtgctgccacctgtggctggaagatccgtggccctgtacgcctctgccgacgaagccagaaatgccctgccccccatccctagagtgatgtggcctcctggattcggcgctgccgagacagtgctggaaggcagcgacggcaccagatttgtgtttggccaccacggcggaagcgagcggcctgctgaaacacaggctggccggcagagaagaaccgccgacgatagggaacacgccctcgagcccgatgactgggaagtgggctgtgaagatgcctgggacagcgaagagggtggcggagatgatggcgacgcccctggcagctccttcggagtgtctatcgtgtctgtggcccctggcgtgctgcgggacagaagagtgggactgaggccagccgtgaaggtggaactgctgagcagctcctccagctccgaggatgaggatgacgtgtggggcggaaggggcggcagaagccctccacagagtagaggatgatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 5 - DNA, 3855 bp, Artificial Sequence
  pGX-5102
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggattctgttcctggtggccgctgccacccgggtgcacagcatgttctgcaccagccccgccaccagaggcgacagcagcgagtctaagcctggcgccagcgtggacgtgaacggcaagatggaatacggcagcgcccctggccccctgaacggcagagatacaagcagaggccctggcgccttctgcacccccggatgggagatccaccccgccagactggtggaagatatcaaccgggtgttcctgtgtatcgcccagagcagcggcagagtgacccgggacagcagacggctgcggcggatctgcctggacttctacctgatgggccggacccggcagaggcctaccctggcctgttgggaggaactgctgcagctgcagcccacccagacccagtgcctgcgggccaccctgatggaagtgtcccacagaccccccagaggcgaggacggcttcatcgaggcccccaacgtgcccctgcaccgcagcgctctggaatgcgacgtgtccgacgacggcggcgaggacgacagcgacgatgatggcagcacccccagcgacgtgatcgagttcagagacagcgacgccgagagcagcgacggcgaggacttcatcgtggaagaggaaagcgaggaatccaccgacagctgcgagcccgatggcgtgcccggcgactgctatagagatggcgacggctgcaacacccccagccccaagaggccccagcgggccatcgaaagatacgctggcgccgagacagccgagtacaccgccgccaaagccctgacagccctgggcgaaggcggcgtggactggaagagaagaaggcacgaggcccctcggcggcacgacattcctccacctcatggcgtgtagtgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 6 - DNA, 4890 bp, Artificial Sequence
  pGX-5103
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggattctgttcctggtggccgctgccacccgggtgcacagcatgggcaccgtgaacaagcccgtcgtgggagtgctgatgggcttcggcatcatcaccggcaccctgcggatcaccaaccccgtgcgggccagcgtgctgagatacgacgacttccacatcgacgaggacaagctggacaccaacagcgtgtacgagccctactaccacagcgaccacgccgagagcagctgggtgaaccggggcgagagcagccggaaggcctacgaccacaacagcccctacatctggccccggaacgactacgacggcttcctggaaaacgcccacgagcaccacggcgtgtacaaccagggcagaggcatcgacagcggcgagcggctgatgcagcctacccagatgagcgcccaggaagatctgggcgacgacaccggcatccacgtgatccccaccctgaacggcgacgaccggcacaagatcgtgaacgtggaccagcggcagtacggcgacgtgttcaagggcgacctgaaccccaagccccagggccagagactgatcgaggtgtccgtggaagagaaccaccccttcaccctgagagcccccatccagcggatctacggcgtgcggtataccgagacttggagcttcctgcccagcctgacctgcaccggggatgccgcccctgccatccagcacatctgcctgaagcacaccacctgtttccaggacgtggtggtggacgtggactgcgccgagaacaccaaagaggaccagctggccgagatcagctaccggttccagggcaagaaagaggccgaccagccctggatcgtggtgaacaccagcaccctgttcgacgagctggaactggacccccccgagatcgagcccggcgtgctgaaggtgctgcggaccgagaagcagtacctgggcgtgtacatctggaacatgcggggcagcgacggcaccagcacctacgccacctttctggtgacatggaagggcgacgagaaaacccggaaccccacccctgccgtgacccctcagcctagaggcgccgagtttcacatgtggaactaccactcccacgtgttcagcgtgggcgacaccttcagcctggccatgcatctgcagtacaagatccacgaggcccccttcgacctgctgctggaatggctgtacgtgcccatcgaccctacctgccagcccatgcggctgtacagcacctgtctgtaccaccccaacgccccccagtgcctgagccacatgaacagcggctgcaccttcaccagcccccacctggctcagagggtggccagcaccgtgtaccagaactgcgagcacgccgacaactacaccgcctactgcctgggcatcagccacatggaacccagcttcggcctgatcctgcacgacggcggcaccaccctgaagttcgtggacaccccagagagcctgagcggcctgtacgtgttcgtggtgtacttcaacggccacgtggaagccgtggcctacaccgtggtgtccaccgtggaccacttcgtgaacgccatcgaggaacggggcttcccacccacagccggacagcctccagccaccaccaagcccaaagaaatcacccccgtgaaccccggcaccagccctctgctgagatatgccgcctggacaggcggactggccgctgtggtgctgctgtgcctggtgatcttcctgatctgcaccgccaagcggatgagagtgaaggcctaccgggtggacaagtccccctacaaccagagcatgtactacgccggcctgcccgtggacgatttcgaggacagcgagagcaccgacaccgaggaagagttcggcaacgccatcggcggcagccacggcggctctagctacaccgtgtacatcgacaagacccgctgatgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt